(19) United States
(12) Patent Application Publication
(10) Pub. No.: US 2006/0269969 A1
(43) Pub. Date: Nov. 30, 2006

(54) Screening for cell-targeting ligands attached to metal nanoshells for use in target-cell killing

(76) Inventor: Kimon Angelides, Houston, TX (US)

Correspondence Address: ERIC P. MIRABEZ, BIOARRY SOLUTIONS LLC, 35 TECHNOLOGY DRIVE, WARREN, NJ, 07059, US

(21) Appl. No.: 11/438,529

(22) Filed: May 22, 2006

Related U.S. Application Data
(63) Continuation of application No. 10/651,343, filed on Aug. 28, 2003.
(60) Provisional application No. 60/406,509, filed on Aug. 28, 2002.

(51) Int. Cl. G01N 33/567; G01N 33/569; G01N 33/554; G01N 33/53

(52) U.S. Cl. 435/7.2; 435/7.31; 435/7.32; 977/900

(57) ABSTRACT

Disclosed is a screening method for detecting cells producing binding agents, including Monoclonal Antibodies, which bind to particular antigens on cells or organisms such that, when the binding agents are bound to said antigens, a nanoshell or metal colloid coupled to the binding agents is capable, upon illumination with a laser or other light source, of killing the cells or organisms. Samples of binding agent-producing cells, which have been subject to limiting dilution so that there is about one cell per well, are placed into the wells of a microtiter plate. Cells or organisms to be killed are added to the wells, as are nanoshells or metal colloids which are treated in a manner appropriate to permit coupling to the binding agents. The wells are illuminated with a light source capable of heating the nanoshells or metal colloids, and the wells containing killed cells or organisms are detected.

### PRIORITY CLAIM

Priority is hereby claimed to U.S. Provisional Application Ser. No. 60/406,509, filed on Aug. 28, 2002.

### BACKGROUND OF THE INVENTION

Solid metal nanoparticles (i.e. metal spheres of uniform composition and nanometer dimensions, for example, those in a colloidal solution) possess the ability to strongly absorb light at certain frequencies. This property is due to the fact that the electrons in the metal become excited upon exposure to light. However, metal colloid solutions will maximally absorb light only at relatively narrow ranges of wavelengths, and these ranges cannot readily be shifted by altering particle size or composition. For example, silver particles 10 nanometers (“nm”) in diameter absorb light maximally at approximately 355 nm while similar sized gold particles absorb maximally at about 520 nm. These absorbance maximums are insensitive to changes in particle size and to changes in dielectric coatings on the particles.

Human blood, cells and tissues minimally absorb certain near-infrared wavelengths of light. Light sources, such as a laser, which deliver such wavelengths, are able to penetrate the body. This could allow illumination of any nanoparticles in blood, cells or tissues.

U.S. Pat. No. 6,344,272 (the “'272 patent”) discusses metal nanoshells, which are homogeneous in size and have a nonconducting inner layer that is surrounded by an electrically conducting material. The ratio of the thickness of the nonconducting layer to the thickness of the outer conducting shell determines the wavelength of maximum absorbance of the particle. Such nanoshells can be designed to absorb in spectra from about 300 nanometers to 20 micrometers.

The '272 patent notes that the reduction of gold onto particles of a less-conductive substrate had been shown to red shift the absorption maximum from 520 nm (for a gold colloid) to between approximately 600 nm and 900 nm, depending on the amount of gold deposited on the core and the size of the core. The '272 patent further notes, however, that such gold-substrate particle diameters are limited to sizes of approximately 40-45 nm with a thin gold shell, typically less than 5 nm, which limits the absorbance maximum to wavelengths no larger than 900 nm. The patent goes on to discuss certain metal nanoshells having absorbance peaks of up to 910 nanometers, and others up to 1010 nanometers. Such spectra are within the infrared range and such particles would be suitable for defined in vivo uses, as they could absorb light from a laser or other external light source.

One use for such metal nanoshells is described in U.S. Pat. No. 6,428,811, which discusses metal nanoshells which absorb infrared light, thereby producing heat. These nanoshells are combined with a temperature-sensitive material including a drug product, to provide an implantable or injectable system for drug delivery, activated to release the drug upon exposure to near-IR light by a laser or other external source.

In a similar manner to the system discussed in U.S. Pat. No. 6,428,811, one could use targeting agents, for example, monoclonal antibodies, coated on metal nanoshells to target or organisms one wishes to kill, e.g., tumor cells, infected cells, parasites or microbes. The monoclonal antibodies would target an antigen (or antigens) on the surface, and thereby bind the nanoshell to such cell or organism. If now illuminated with a light source, the heat emitted by the nanoshell would kill the cell or organism it was bound to (provided the nanoshell was sufficiently heated to reach a lethal temperature).

With this approach, it can be difficult to select a suitable monoclonal antibody. In the conventional process for making monoclonal antibodies, a mouse is immunized with an antigen of interest, and its immune system is boosted with adjuvants so that it generates an enhanced response against the immunogen. The mouse B lymphocytes are extracted from the mouse spleens (which contain high numbers of B-lymphocytes), and then fused with an immortal myeloma cell line. Some of the hybridomas resulting from the fusion produce monoclonal antibodies to the antigen initially used to immunize the mouse. The single hybridoma (or hybridomas) among this sea of fused cells secreting antibody into the medium is then screened, and those with the desired characteristics are selected.

In view of the millions of different antibody-secreting cells in the hybridoma pool, selection is usually a labor-intensive trial and error process, the success of which is dependent on the screening method, the skill with which it is applied and the characteristics of the antibody sought. Conventional methods of screening involve one or more of enzyme linked immunosorbent assays (ELISA), Western blotting, dot blots and others. Oftentimes, no monoclonal antibodies targeting a particular antigen can be found using conventional methods, or, those that are found are of low affinity or specificity, or have undesirable and unanticipated effects for therapeutic use.

The targeting agent (whether a monoclonal antibody or other) for a metal nanoshell may be particularly difficult to isolate, as it must be able to bind to an antigen which is in relatively close proximity to the cell surface and/or, have a conformation or characteristics permitting the nanoshell to get close to the cell surface. Certain surface antigens, including some glycoproteins, can extend from the surface. In the event the targeting agent binds such “distant” antigens in vivo, the nanoshell may not be close enough to the target to kill it upon illumination and heating. Other antigens, due to conformation or other characteristics, may also not be suitable.

It would advantageous to be able to select only the targeting agents to those antigens which, when bound to a nanoshell, allow nanoshells relatively close to the surface, as such targeting agents are the ones useful in cell killing.

### SUMMARY OF THE INVENTION

The invention includes methods for selection of agents targeting particular cell surface (or organism surface) antigens. The agents are suitable for in vivo targeting of a nanoshell to those antigen(s) that are close enough to the surface to be inactivated or that kill cells when the nanoshell is heated by illumination from an external light source, for example a laser. The agents selected, therefore, are suitable for use in therapeutic products where metal nanoshells are illuminated with a light source in the infrared spectra to kill cells or organisms, including, for example, tumor cells, infected cells, parasites and microbes.

To achieve these advantages, cells producing (or which may be producing) monoclonal antibodies or related binding agents to the target cells are placed in separate wells of a multi-well microtiter plate. Next, the target cells are placed in each well, together with metal nanoshells which have been coated with protein G (which binds to the Fc region of a monoclonal antibody) or with another binding agent, preferably a protein, capable of binding to the coated nanoshells: (i) to monoclonal antibodies if the cells in the wells are producing monoclonal antibodies; or (ii) to the binding agent if the cells in the wells are producing such agent. Alternatively, the components can be mixed separately on line, where samples are taken from the wells of the microtiter plate containing candidate binding agents for testing of nanoshell-mediated antigen inactivation or cell killing, and protein G or another substance capable of binding to the coated nanoshells.

The plates are then illuminated with a light source, preferably a laser, at a wavelength which is absorbed by the nanoshells and causes heating to a lethal temperature. The protein G coating (or another coating, if substituted for protein G) of the nanoshell therefore binds the nanoshell to the Fc on the antibodies (in one case, or to the binding agents, in the other case) which are bound to the target cells. Those target cells that have surface antigens to which the antibody-coated nanoshell binds, or which are of a type or conformation such that the nanoshell is in relatively close proximity to the cell, will be inactivated or killed by the heating effect. Other target cells, including those without such antigens and those that have not been bound by antibodies or binding agents in the wells, will remain essentially, intact. However, it is noted that Brownian motion of the unbound nanoshells in the wells may place some close enough to a target cell to kill it, even though it is not bound to it. But it is believed that this effect will not result in death of enough cells to significantly affect assay results.

The wells having significant numbers of killed target cells can be differentiated by well-known means, such as by addition of a dye to all the wells, where the dye fluoresces upon contact with components released by lysed (killed) cells. The antibodies in those wells containing cells producing antibodies (or other binding agents) that bind to target antigens close to the target cell surface, are appropriate for use for in vivo cell killing. The cells from such wells are cloned, and the high-producing cell lines are selected and used to produce therapeutic candidates.

### BRIEF DESCRIPTION OF THE DRAWINGS

FIG. 1 schematically depicts nanoshell coupled to an antibody and bound to the surface of a tumor cell.

FIG. 2 schematically depicts a multi-well microtiter plate illuminated by a laser.

FIG. 3 depicts a single well of a multi-well microtiter plate containing within the well nanoshells, antibody-producing cells, antibodies and tumor cells.

### DETAILED DESCRIPTION OF THE INVENTION

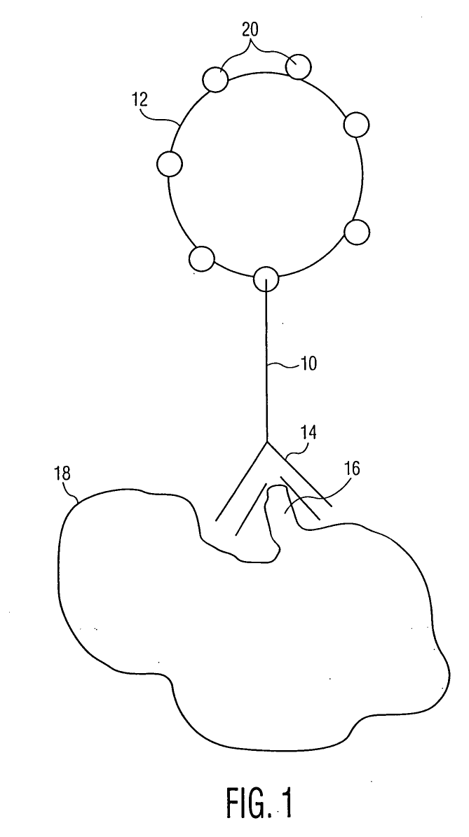

FIG. 1 shows a nanoshell 12 coated with protein G (20 in the figure) coupled to the Fc region of an antibody 10, which is bound at its antigen-binding portion 14 to an antigen 16 of a cell 18. Cells 18 can preferably be tumor or infected cells, but could also be cells from parasites or infectious microbes, depending on which of the foregoing one is screening antibodies in the wells for. Antibody 10 is preferably a monoclonal antibody. Nanoshell 12 can be any of a number of nanoshells, which generate heat upon illumination, some examples of which are described in U.S. Pat. Nos. 6,428,811 and 6,344,272 (incorporated by reference). As a substitute for a nanoshell, one could also use conventional metal colloids, e.g., gold, as described in the “Background” section of U.S. Pat. No. 6,428,811. Conventional colloids will also generate heat when illuminated, though not as efficiently as nanoshells.

Coupling the nanoshell 12 to the antibody 10 in FIG. 1 by coating of the nanoshell with protein G is merely one of several ways to accomplish joinder. One could also simply absorb the antibody to the metal nanoshell. One limitation is that the method of coupling must not affect the binding characteristics of antibody 10, so it is preferable to couple the antibody to the nanoshell 12 through the antibody Fc portion, which is distal from the antigen-binding portion and less likely to affect antigen binding. In the event one is using a binding agent (protein) other than an antibody 10, one would want to ensure that the binding to the metal nanoshell does not affect binding to the target cell.

It is also possible to use certain antibody fragments, including Fab, Fab′ and F(ab′)₂, instead of a whole antibody. The Fab fragment also contains the constant domain of the light chain and the first constant domain (C_{H 1}) of the heavy chain. Fab′ fragments differ from Fab fragments by the addition of a few residues at the carboxy terminus of the heavy chain C_{H 1} domain including one or more cysteines from the antibody hinge region. F(ab′)₂ antibody fragments are pairs of Fab′ fragments covalently bound through their hinge region cysteines. If using such fragments with the system of the invention, the coupling to the nanoshell must not interfere with antigen binding; that is, such coupling would normally be located in the heavy chain region (such as one exists) of the fragment.

The making of monoclonal antibodies and fragments is described below.

Making Monoclonal Antibodies

Monoclonal antibodies suited for use in the invention can be isolated from continuous hybrid cell lines formed by the fusion of antigen-primed immune lymphocytes with myeloma cells. Monoclonal antibodies for use in the present invention can also be obtained from lymphocytes or hybridomas made from lymphocytes of patients with disease conditions, including infectious, viral, cancer, and autoimmune diseases. The monoclonal antibodies selected must bind to surface antigens of the tumor cells, infected cells, parasites or microbes one is attempting to kill. These antibodies are obtained through screening (see, for example, the discussion below).

Monoclonal antibodies are highly specific, being directed against a single antigenic site. Furthermore, in contrast to conventional antibody (polyclonal) preparations which typically include different antibodies directed against different determinants (epitopes), each monoclonal antibody is directed against a single epitope on the antigen. Monoclonal antibodies can therefore be selected to bind to specific antigens on the cell surface, and further only to specific epitopes on such antigens. The hybridoma technique described originally by Kohler and Milstein, Eur. J. Immunol. 6, 511 (1976) can be used to produce hybrid cell lines (“hybridomas”) that secrete high levels of monoclonal antibodies against many specific antigens

In making monoclonal antibodies, there is a relatively well-established route and schedule of immunization of a host animal which stimulates antibody-producing lymphoid B cells, and thus antibody production, by the host animal. A preferred host animal is a mouse, but other animals can be used.

In a conventional immunization of a host animal, following immunization, B cells are fused with myeloma cells (immortal tumor cells derived from plasma cells) to generate an immortal hybrid cell line. These hybridoma cell lines can be cultivated and sub-cultivated indefinitely, to produce large quantities of monoclonal antibodies. The B cells used are taken either from lymph node tissue or spleen tissue of immunized animals. Spleen cells generally offer a mores concentrated and convenient source of antibody producing cells when the host is a mouse.

It is possible to fuse cells of one species with another. However, it is preferred that the source B cells and myeloma is the same species.

The hybrid cell lines can be maintained in culture in vitro in cell culture media. The cell lines of this invention can be selected and/or maintained in hypoxanthine-aminopterin-thymidine (HAT) medium. In fact, once the hybridoma cell line is established, it can be maintained on a variety of nutritionally adequate media. Moreover, the hybrid cell lines can be stored and preserved in any number of conventional ways, including freezing and storage under liquid nitrogen. Frozen cell lines can be cultured indefinitely and then revived to resume synthesis and secretion of monoclonal antibody. The secreted antibody is recovered from tissue culture supernatant by conventional methods such as precipitation, ion exchange chromatography, affinity chromatography, or the like.

Making Less Immunogenic Antibodies and Fragments

Because the antibodies being screened for are intended for use in therapy, the antibodies screened for would preferably be chimeric, deimmunized, humanized or human antibodies, or antibody fragments. Such antibodies have substantial human-derived regions, and can reduce immunogenicity and thus avoid human anti-mouse antibody (HAMA) response. It may be desirable to adjust, following generation of hybridomas, the antibody effector function, such as by altering the constant regions to human IgG1, IgG3 constant regions, as such antibodies will augment antibody-dependent cellular cytotoxicity (S. M. Canfield and S. L. Morrison, J. Exp. Med., 1991: 173: 1483-1491) and complement mediated cytolysis (Y. Xu et al., J. Biol. Chem., 1994: 269: 3468-3474; V. L. Pulito et al., J. Immunol., 1996; 156: 2840-2850) and may aid in cell-killing when used in therapy.

Chimeric antibodies are produced by recombinant processes well known in the art, and have animal variable regions and human constant regions. Humanized antibodies have more human peptide sequences than do chimeric antibodies. In a humanized antibody, only the complementarity determining regions (CDRs) which are responsible for antigen binding and specificity are animal derived and have an amino acid sequence corresponding to the animal antibody, and substantially all of the remaining portions of the molecule (except, in some cases, small portions of the framework regions within the variable region) are human derived and correspond in amino acid sequence to a human antibody. See L. Riechmann et al., Nature, 1988; 332: 323-327; G. Winter, U.S. Pat. No. 5,225,539; C. Queen et al., U.S. Pat. No. 5,530,101.

Deimmunized antibodies are antibodies in which the T and B cell epitopes have been eliminated, as described in International Patent Application PCT/GB98/01473. They have reduced immunogenicity when applied in vivo.

U.S. Pat. No. 4,946,778 describes the making of single peptide chain binding molecules in which the heavy and light chain Fv regions are connected Fab can be constructed and expressed by similar methods (M. J. Evans et al., J. Immunol. Meth., 1995; 184: 123-138), as can Fab′ and F(ab′)₂.

Human antibodies may be most preferred for therapy. One technique to generate them is to extract antibody-producing cells from humans, and use well-known techniques, such as EBV immortalization of B cells, to generate human hybridomas. Human antibodies can also be made by several other methods, including by use of human immunoglobulin expression libraries (Stratagene Corp., La Jolla, Calif.) to produce fragments of human antibodies (VH, VL, Fv, Fd, Fab, or F(ab′)₂, and using these fragments to construct whole human antibodies using techniques similar to those for producing chimeric antibodies. Human antibodies can also be produced in transgenic mice with a human immunoglobulin genome. Such mice are available from Abgenix, Inc., Fremont, Calif., and Medarex, Inc., Annandale, N.J. Briefly, this approach involves disruption of endogenous murine heavy and k light chain loci, followed by construction of heavy and light chain transgenes containing V, D, J segments, and C genes of human origin. These are then introduced by pronuclear microinjection using human transgenes. The mice are then cross-bred to generate the human antibody producing strains. This technique is describe in more detail in, among other references, U.S. Pat. No. 5,569,825 (incorporated herein by reference).

All of the wholly and partially human antibodies, and the fragments, are less immunogenic than wholly murine MAbs. All are therefore less likely to evoke an immune or allergic response. Consequently, they are better suited for in vivo, administration in humans than wholly animal antibodies, especially when repeated or long-term administration is necessary. In addition, the smaller size of an antibody fragment may help improve tissue bioavailability, which may be critical for better dose accumulation in acute disease indications.

Applying the Screening Process of the Invention

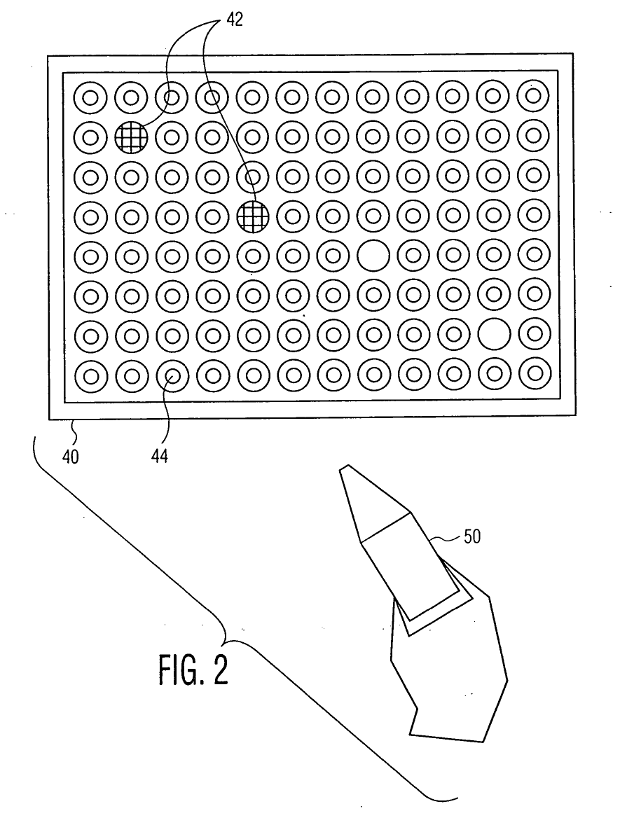

FIG. 2 depicts a 96 well microtiter plate 40 with fluorescent wells 42 and other wells designated as 44, wherein wells 42 are shaded to indicate that they has changed color and/or are fluorescing. Rather than microtiter plate 40, one can use any of a number of well-known multi-well microtiter plates used in cell screening assays, many of which have more wells.

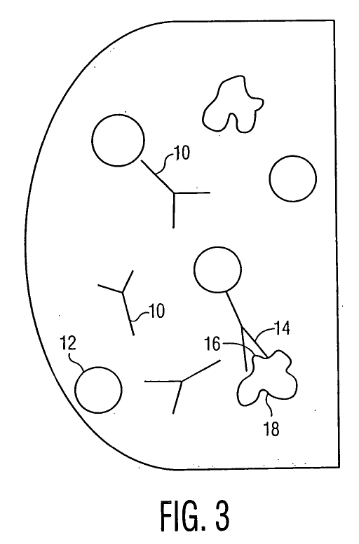

A laser 50 is shown illuminating a well in microtiter plate 40. FIG. 3 depicts one of the wells of the microtiter plate 40 having in it antibodies 10. As noted above, antibodies 10 are preferably a monoclonal antibody or fragment thereof, having an Fc portion suited for binding to the nanoshell.

Some antibodies 10 are shown coupled to nanoshells 12, and some such complexes in turn are shown bound to a surface cell antigen 16 of a cell 18. Upon heating nanoshell 12 by illumination with laser 10, the cell 18 will lyse and release its contents into the well environment.

Illuminating individual wells of a microtiter plate 40 causes cell death which can be detected by well-known techniques such as LIVE/DEAD (Molecular Probes), Almar Blue, nuclear stains, and cell viability metabolic stains. The cells one is interested in killing in the functional assay (infected cells or tumor cells) with a fluorescent dye, such as propidium iodide or EthD-1 which enters cells with damaged membranes and undergoes a 40-fold enhancement of fluorescence upon binding to nucleic acids, thereby producing a bright red fluorescence in dead cells (ex/em.about.495 nm/.about.635 nm). EthD-1 is excluded by the intact plasma membrane of live cells. Another suitable marker is Almar Blue, which fluoresces if the cell is active. Alternatively, one can use the two color reporter system available from Molecular Probes, Inc. of Eugene, Oreg. The cell-permeant esterase substrate calcein AM is nonfluorescent until converted by enzymatic activity to highly fluorescent calcein, which is retained within live cells and imparts an intense green fluorescence. Ethidium homodimer-1 undergoes a fluorescence enhancement upon binding nucleic acids, producing a bright red fluorescence. This dye is excluded from cells that have intact plasma membranes but is readily able to enter dead cells. Thus, live cells fluoresce green, while dead cells fluoresce red. In this method, following illumination, the plates are rinsed in PBS calcein/propidium iodide labeling solution is added to the wells. The wells are then analyzed by fluorescent microscopy. Random fields are photographed and 100 nuclei are counted for cell death analysis. Alternatively, fluorescence activated flow cytometry, also known as fluorescence activated cell sorting (“FACS”), can be used to quantitate the number of live or dead cells killed by antibody-coated nanoshells. FACS allows efficient counting of particular cells from large numbers of cells, based on selected characteristics. The live/dead reports on the ability of the antibody, with the bound nanoshell, of killing cells or inactivating cellular antigens.

Similarly, if one was trying to find antibodies to antigens on parasites or infectious microbes, they would be added to the wells of microtiter plate 40 instead of cells 18, or to a flow cytometer and one could screen for them essentially the same way, except one would detect killing of the parasites or microbes by ethidium bromide.

Using the methods of the invention, therefore, allows one to screen out the cells in the particular wells in the microtiter plate 40 or from a microtiter plate which produces antibodies which bind to cell surface antigens and which permit the coupled nanoshell close enough to the cell surface to kill the cell upon illumination with a laser. Wells of a microtiter plate can also be sampled and submitted for analysis using flow cytometry and automated sampling. These are the antibodies best-suited for in vivo use in cell (or organism) killing applications using external illumination of nanoshells. Such antibodies will bind to the same antigens in vivo and permit the nanoshell to get close enough to the cell surface to effect cell death upon external laser illumination. This screening method is therefore extremely useful for all applications where antibodies (or fragments) are to used as targeting agents for nanoshells.

Example of Antibody Selection Using the Screening Process

Following a fusion or production of a large number of cells producing monoclonal antibodies (or fragments) the cells are subject to limiting dilution, and a volume of sample is added to each well of a microtiter plate such that there is about one cell per well. One then adds nanoshells (or colloids, if preferred) which have been treated so as to be coated on their surface with protein G. Protein G will bind to the Fc regions of antibodies. One also adds to the wells the cells or organisms one is ultimately interested in killing, when antibodies selected by these methods are used in a therapeutic application.

The next step is to illuminate the wells with light, preferably from a laser, at frequencies which excite and heat the nanoshells or colloids. The heating effect will kill the cells or organisms which are bound to nanoshells or colloids in relatively close proximity to the cell or organism. The death of the cells is detected by a color change in the well. Cells from the wells in which such cell death occurs are then extracted from the wells, grown in a suitable medium, cloned and if desired subcloned to optimize them as antibody-producing cell lines. The monoclonal antibodies or fragments produced by these cells are then tested in various functional assays and in vivo tests to determine their suitability in therapeutic cell killing applications.

Defined Terms; Claim Scope

As used in the claims below, the term “Monoclonal Antibody” or “Monoclonal Antibodies” refers to all monoclonal antibodies and derivatives and fragments thereof having binding activity, including but not limited to mouse, humanized, human, and Deimmunised antibodies, and fragments including Fab, F(ab′)₂, and Fd fragments, and single chain Fv binding molecules. It should be understood that the terms, expressions and descriptions set forth above are exemplary only and not limiting, and that the invention is defined only in the claims which follow and includes all equivalents of the subject matter of such claims.

1. A screening method for detecting cells producing binding agents, including Monoclonal Antibodies, which bind to particular antigens on cells or organisms such that, when the binding agents are bound to said antigens, a nanoshell or metal colloid coupled to the binding agents is capable, upon illumination with a laser or other light source, of killing the cells or organisms, comprising: placing samples of binding agent-producing cells, which have been subject to limiting dilution so that there is about one cell per well, into the wells of a microtiter plate; adding to the wells, cells or organisms to be killed; adding to the wells, nanoshells or metal colloids which are capable of coupling to the binding agents and providing conditions to effect such coupling; illuminating the wells with a light source capable of heating the nanoshells or metal colloids; and detecting the wells containing killed cells or organisms.

2. The method of claim 1 further comprising extracting the binding agent-producing cells from the wells containing killed cells or organisms.

3. The method of claim 2 further comprising growing the binding agent-producing cells.

4. The method of claim 1 wherein the binding agent is a protein.

5. The method of claim 1 wherein the cell or organisms to be killed are tumor cells, infected cells, parasites or infectious microbes.

6. The method of claim 1 wherein the nanoshells or metal colloids are treated with protein G.

7. The method of claim 1 wherein the light source is a laser emitting at a range of frequencies suitable to heat the nanoshells or metal colloids in the wells.

8. The method of claim 1 wherein the wells with killed cells or organisms are detected by propidium iodide staining techniques.

9. A screening method for detecting cells producing binding agents, including Monoclonal Antibodies, which bind to particular antigens on cells or organisms such that, when the binding agents are bound to said antigens, a metal nanoshell which absorbs in the spectra from about 300 nanometers to 20 micrometers and which is coupled to the binding agents is capable, upon illumination with a laser or other light source, of killing the cells or organisms, comprising: placing samples of binding agent-producing cells, which have been subject to limiting dilution so that there is about one cell per well, into the wells of a microtiter plate; adding to the wells, cells or organisms to be killed; adding to the wells, nanoshells which are capable of coupling to the binding agents and providing conditions to effect such coupling; illuminating the wells with a laser emitting a frequency within the range at which the nanoshell absorbs maximally; and detecting the wells containing killed cells or organisms.

10. The method of claim 9 wherein the nanoshell is designed to absorb maximally at frequencies from 500 to 1010 nanometers and the laser emits at the frequency of maximal absorption.

11. The method of claim 9 further comprising extracting the binding agent-producing cells from the wells containing killed cells or organisms.

12. The method of claim 9 further comprising growing the binding agent-producing cells.

13. The method of claim 9 wherein the cell or organisms to be killed are tumor cells, infected cells, parasites or infectious microbes.

14. The method of claim 9 wherein the binding agent is a protein.

15. The method of claim 9 wherein the nanoshells or metal colloids are treated with protein G.

16. The method of claim 9 wherein the wells with killed cells or organisms are detected by propidium iodide staining techniques.
